(19) United States
(12) Patent Application Publication
(10) Pub. No.: US 2026/0115418 A1
(43) Pub. Date: Apr. 30, 2026

(54) REINFORCED CATHETER DESIGN WITH IMPROVED FLEXIBILITY

(71) Applicant: Toro Neurovascular, Inc., Irvine, CA (US)

(72) Inventors: Hyung Posalit, Dana Point, CA (US); Ryan Walker, Costa Mesa, CA (US)

(73) Assignee: Toro Neurovascular, Inc., Irvine, CA (US)

(21) Appl. No.: 18/926,031

(22) Filed: Oct. 24, 2024

(51) Int. Cl. A61M 25/00

(52) U.S. Cl. CPC A61M 25/0045; A61M 25/0052; A61M 25/0054; A61M 2025/0047; A61M 2025/0059; A61M 2205/0216; A61M 2205/0233; A61M 2205/32

(57) ABSTRACT

A catheter body has a lubricious polymeric inner liner and a composite wall. The composite wall includes a reinforcement structure and an outer jacket. The reinforcement structure has at least two layers of metallic wires, with each layer of metallic wires wound in helical patterns, with the first layer of metallic wires wound around the inner liner and the second layer of metallic wires wound around the first layer of metallic wires, and so on. The outer jacket that is comprised of a distal jacket material that is a soft elastomer of hardness 60 Shore A or lower.

### BACKGROUND OF THE INVENTION

### 1. Field of the Invention

The present invention relates to catheters, and in particular, to materials and a construction for a catheter body that provides improved bendability and kink resistance.

### 2. Description of the Prior Art

Current catheters utilized in neurovascular access and therapeutic procedures exhibit tip bending behavior in which the tip bend initiation site of the leading (or distal) tip does not start immediately at, or close enough to, the distal tip of the catheter. Rather, there is a region of the distal tip that is relatively inflexible and the majority of tip bending initiation occurs some distance, usually about 10 mm-20 mm or more, proximal to the distal tip, which makes it difficult for the catheter to conform to and follow the bends in some vessels, particularly tight bends and bends in small vessels.

This bending problem is illustrated in FIGS. 1A-1C. FIG. 1A shows a conventional catheter C encountering the bend of an arterial pathway. As shown in FIG. 1B, the distal section D of the catheter C remains straight so that the catheter's bend point B occurs after the straight segment of the distal section D. FIG. 1C shows that the distal section D of the catheter C remains straight even after navigating the bend. It can be appreciated from FIGS. 1A-1C that it would be more difficult to navigate a bend that has a greater angle than that shown in FIGS. 1A-1C.

The tips of some catheters are also not sufficiently atraumatic because they are relatively hard. Therefore, when the catheter tip fails to bend at a bend in a vessel, vessel dissections and perforations can occur.

Additional issues with existing catheters include: (1) poor kink resistance and (2) low flexibility. If such catheters are deformed or kinked, they may not return to their original, straight orientation, which makes it difficult to insert the catheter farther into a vessel. Inelasticity and low flexibility lead to the bending and trauma issues discussed above.

Thus, there remains a need for an improved catheter body that addresses the drawbacks discussed above.

### SUMMARY OF THE DISCLOSURE

It is an object of the present invention to provide a catheter body that provides improved bendability adjacent its distal end.

The present invention is directed to single-lumen catheters intended for vascular access and therapeutic procedures, such as thrombectomy via blood clot aspiration. These catheters provide enhanced flexibility and kink resistance and improved bending behavior near the distal tip of the catheter.

More specifically, the present invention provides a single catheter assembly for use in accessing or treating locations in the tortuous arterial anatomy, which includes but is not limited to the cerebral circulation.

To meet the objectives of the present invention, there is provided a catheter body having a lubricious polymeric inner liner and a composite wall. The composite wall includes a reinforcement structure and an outer jacket. The reinforcement structure has at least two layers of one or more metallic wires, with each layer of metallic wires wound in helical patterns, with the first layer of metallic wires wound around the inner liner and the second layer of metallic wires wound around the first layer of metallic wires, and so on. The outer jacket that is comprised of a distal jacket material that is a soft elastomer of hardness 60 Shore A or lower.

### BRIEF DESCRIPTION OF THE DRAWINGS

FIGS. 1A-1C illustrate the bending behavior for the distal end of a conventional catheter body, and FIGS. 1D-1F illustrate the bending behavior for the distal end of catheter body of the present invention.

FIG. 2 is an exploded schematic view of a catheter body according to one embodiment of the present invention.

FIG. 3 is a longitudinal cross-sectional view of the catheter body of FIG. 2.

FIG. 4A is a schematic view of only the inner liner and one metallic layer for the catheter body of FIG. 2.

FIG. 4B is an exploded schematic view of FIG. 4A.

FIG. 5A is a flattened view of only the inner layer and the metallic layers that make up the reinforcement structure for the catheter body of FIG. 2.

FIG. 5B is a flattened view of the reinforcement structure for the catheter body of FIG. 5A showing only a single wire visible from each parallel group of wires in the second layer.

FIG. 5C is a flattened view of the reinforcement structure for the catheter body of FIG. 5A showing only two wires visible from each parallel group of wires in the second layer.

### DETAILED DESCRIPTION OF THE PREFERRED EMBODIMENTS

The following detailed description is of the best presently contemplated modes of carrying out the invention. This description is not to be taken in a limiting sense, but is made merely for the purpose of illustrating general principles of embodiments of the invention. The scope of the invention is best defined by the appended claims.

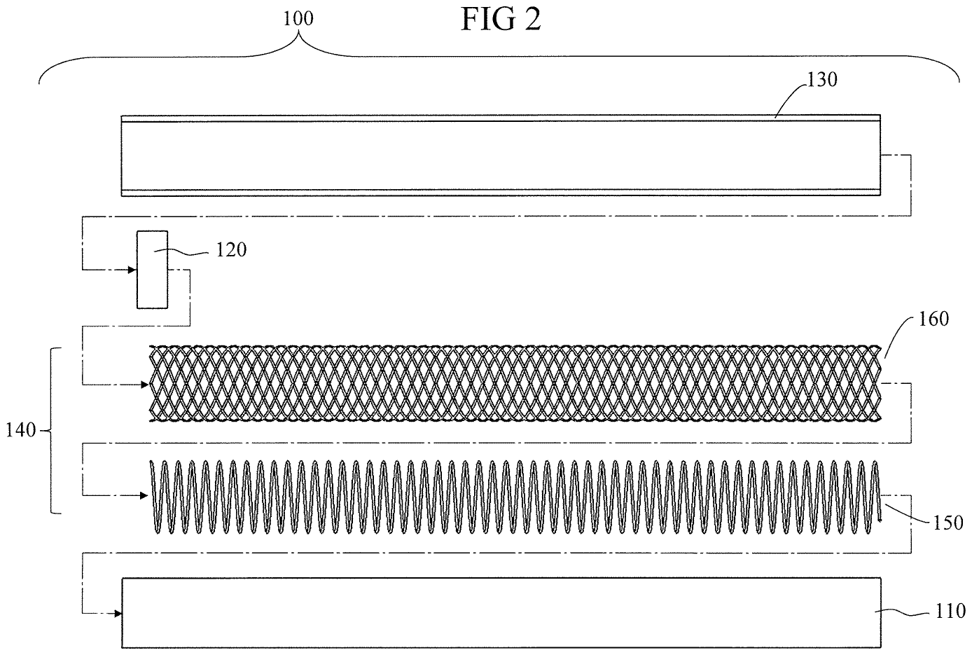

Referring now to FIGS. 2-5, the catheter body 100 of the present invention has an inner liner 110 and a composite wall. The composite wall includes a reinforcement structure 140, a marker band 120 and an outer jacket 130 which encapsulates the reinforcement structure 140 and the marker band 120. The reinforcement structure 140 in turn is made up of a plurality of metallic layers which are wound in helical patterns, first around the inner liner 110 and then around each subsequent metallic layer. The catheter body 100 has a distal region 102 which extends for a length of 3 cm to 30 cm from the distal end of the catheter body 100 and is comprised of the same components above.

Referring to FIGS. 2-4, the inner liner 110 is the inner-most component of the catheter body and defines the inner lumen 112 for the catheter body 100. The inner liner 110 can be embodied in the form a thin-walled tube made of a low-friction material such as PTFE or other polymer with lubricious additives. The inner liner 110 extends from the proximal end of the catheter to the distal end of the catheter body 100.

The reinforcement structure 140 is made up of a plurality of metallic layers which are wound in helical patterns, first around the inner liner 110 and then around each subsequent metallic layer. In one embodiment, the reinforcement structure 140 has a first metallic layer 150 that is wound around the inner liner 110 and a second metallic layer 160 that is wound around the first layer 150. The layers 150 and 160 are made up of metallic wires that are wound in helical patterns. The wires used may be stainless steel or a shape memory alloy such as a nickel-titanium alloy, and may be round, oval, rectangle, square, triangular, crescent, or a combination thereof, in cross-sectional shape. All wires of the reinforcement structure 140 extends from the proximal end of the catheter body 100 to a position offset proximally from the distal tip, as described in greater detail below in connection with the marker band 120.

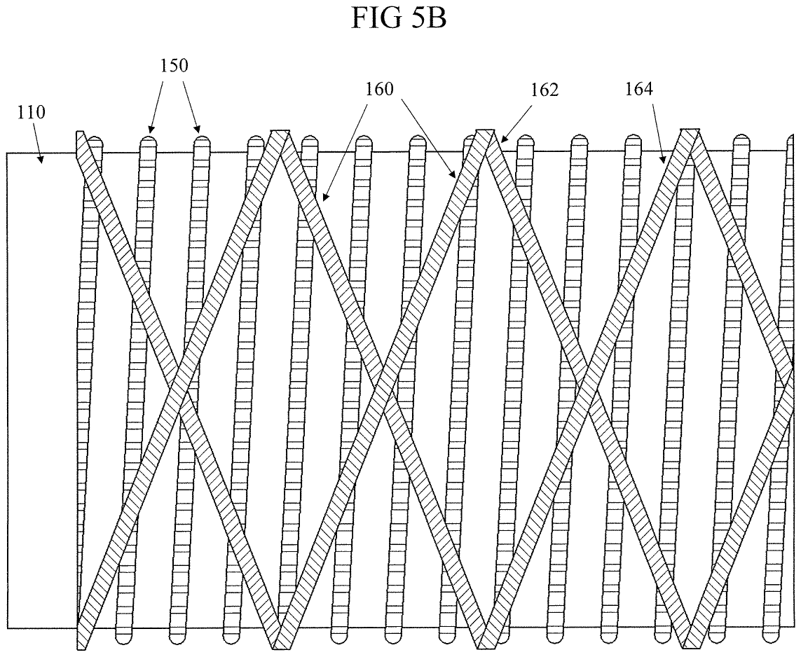

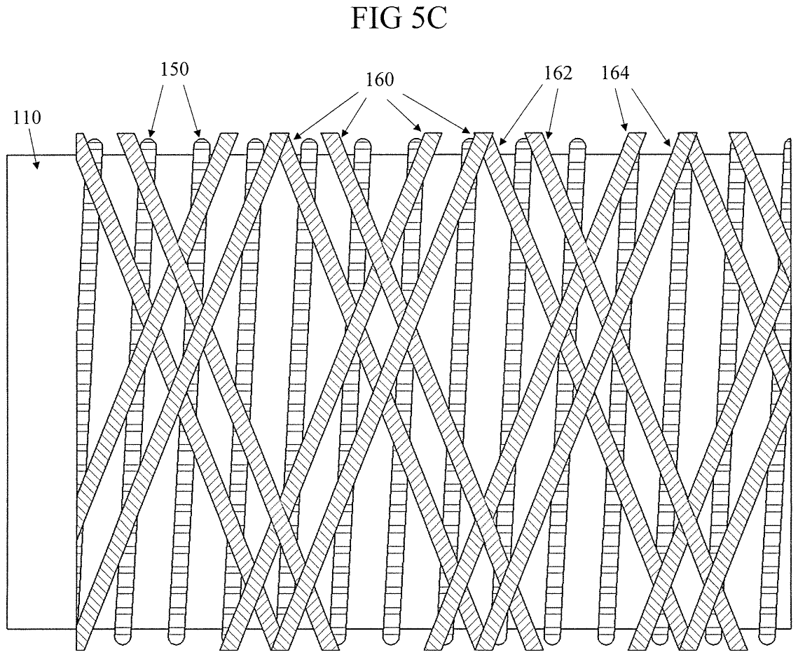

The word “layer” is used to describe the layers 150 and 160, which together make up the reinforcement structure 140. Each metallic layer may be made up of one or more wires that are wound in a helical pattern. In this regard, one or more wires that are wound onto the assembly at the same radial distance from the inner liner 110 may be described as being in the same “layer” of the reinforcement structure 140. In the preferred embodiment, the first metallic layer 150 is made up of a single wire wound in a helical pattern around the inner liner 110 and the second metallic layer 160 is made up of multiple wires wound around the first layer 150, where half of the wires of the second metallic layer 160 are wound in a clockwise helical pattern and the other half are wound in a counter-clockwise helical pattern, with the opposing groups of wires passing over-and-under one another to be inter-woven so as to result in the same layer. This is best shown in FIGS. 5A-5C.

Furthermore, a primary characteristic of the reinforcement structure may be defined as the “Wire Coverage Ratio”, which describes the percentage of the surface area of the inner liner 110 which is covered by all the layers of the reinforcement structure 140. The term “Parallel Groups” can be defined as all wires that are wound in the same direction and at the same angle such that they never intersect, regardless of which layer they are in. In this regard, wires from different layers can be in the same parallel group. This is because “layers” is a term related to the construction while “Parallel Groups” is related to the calculation. Since the wires are parallel, they will never cross over each other, so they are treated as a group for the calculation of Wire Coverage Ratio (see below).

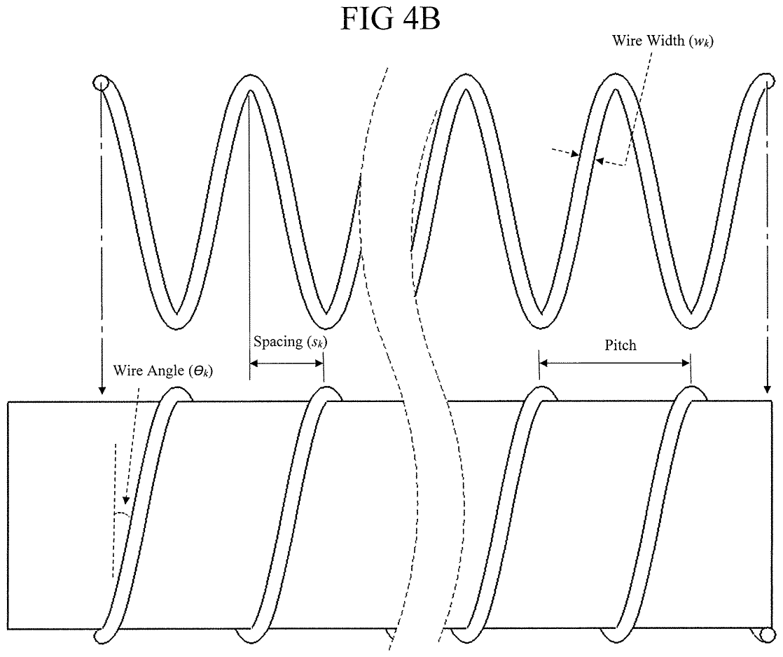

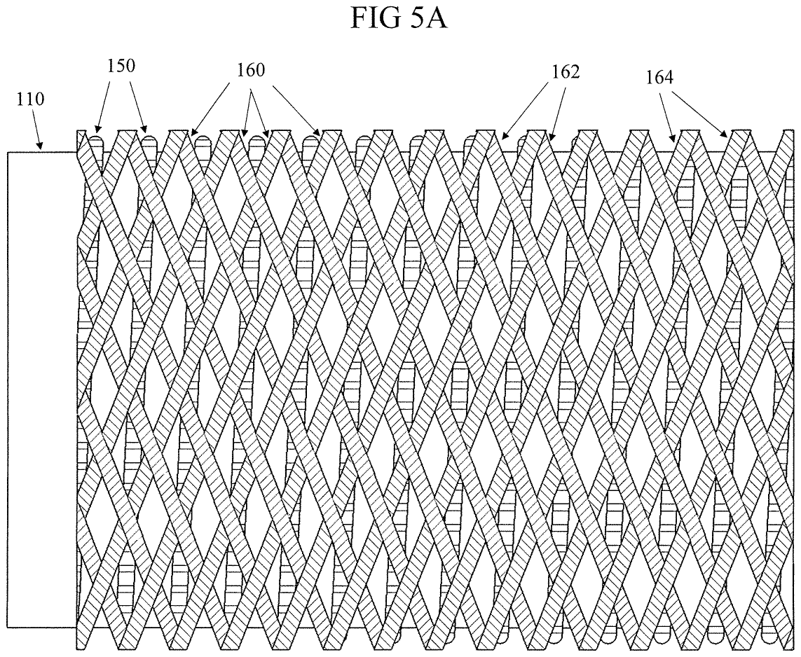

FIG. 4A best shows an example of the inner liner 110 and one metallic layer 150. In this example, the metallic layer 150 is comprised of only one parallel group, which in turn is comprised of two wires. FIG. 4B shows the same example with one wire exploded from the assembly while the second wire remains on the assembly to show the independence of the two wires that comprise the single parallel group and single layer 150. In other embodiments, the first metallic layer 150 may be comprised of one wire, two wires, or more than two wires.

FIG. 5A is a flattened view that best shows an example of the inner liner 110 and two metallic layers 150 and 160. The first layer 150 is a single parallel group comprised of one wire wrapped around the inner liner 110. The second layer 160 is comprised of two parallel groups 162 and 164. Parallel group 162 is wrapped around the first layer 150 in a clockwise helical pattern, while parallel group 164 is wrapped around the first layer 150 in a counter-clockwise helical pattern, and parallel groups 162 and 164 are woven over-and-under one another so as to comprise the single layer 160. The parallel groups 162 and 164 are each comprised of four wires. FIG. 5B shows how multiple wires make up each parallel group by showing the same example from FIG. 5A with only a single wire visible from each parallel group 162 and 164. FIG. 5C shows the same example with two wires visible from each parallel group 162 and 164. Following this construction, it can be seen that four wires comprise each parallel group 162 and 164 shown in FIG. 5A for a total of eight wires in layer 160. In other embodiments, the second metallic layer 160 may be comprised of 5 or more wires but is always comprised of two inter-woven parallel groups.

The Wire Coverage Ratio is then calculated as the summation of the linear coverage percentage by each Parallel Group of wires, as defined in the equation below. See FIG. 4 for reference to the variables.

$$W C {R}_{n} = {\sum }_{k = 1}^{n} ( \frac{{w}_{k}}{( cos {θ}_{k} ) ( {s}_{k} )} ) ( 100 \% - W C {R}_{( k - 1 )} )$$

Where:
WCR=Wire Coverage Ratio
n=number of distinct Parallel Groups of wires in the reinforcement structure 140
WCRₙ=The combined Wire Coverage Ratio of all n Parallel Groups of wire(s) of the reinforcement structure 140
wₖ=the wire width of wire(s) in the k^{th} Parallel Group (shown in FIG. 4)*
θₖ=the angle of wire(s) in the k^{th} Parallel Group (shown in FIG. 4), as measured relative to a cross-section of the catheter that is parallel to the catheter end
sₖ=the spacing between consecutive wires in the k^{th} Parallel Group (shown in FIG. 4)*. This is not to be confused with the pitch, which may be used to define the length for a single wire to repeat. For a Parallel Group with only a single wire, the pitch and the spacing are the same.
WCR₀=0%
*if various wires in a single Parallel Group have different widths wₖ, or if the spacing sₖ is variable, a more complex formula should be used to represent the wire width or spacing when calculating the percentage of coverage over a particular region of the catheter.

Conventional knowledge and designs show that a significantly high Wire Coverage Ratio approaching or above 50% may result in high hoop strength and kink resistance, but also high bending stiffness. The present invention utilizes multiple layers of very small wires (i.e., smaller wire cross-sectional area, including with widths wₖ and wire thicknesses) relative to the catheter diameter, at very small angles, to enable very low bending stiffness while increasing the Wire Coverage Ratio above 50% to maintain the high hoop strength and kink resistance benefits in the distal region 102.

In order for the present invention to achieve low bending stiffness while maintaining the high hoop strength and kink resistance enabled by such a high WCR in the distal region 102, each layer of wires is constructed in such a way to utilize sufficiently small wires at low wire angles θₖ as defined below.

For metallic reinforcement layers containing five or more wires, the wire angle θₖ must be less than 18 degrees in the distal region 102. The size of the wires must be such that the Wire Size Ratio (WSR) is less than 0.7%. Here, “Wire Size Ratio” is defined as the summation of the cross-sectional area of all wires in a single layer divided by the cross-sectional area of the catheter inner diameter. For example, a catheter with inner diameter 0.088″ may have a layer of 16 wires, each having a rectangular cross section with 0.001″×0.002″ dimensions. The summation of the cross-sectional area of all wires is then 0.000032 sq. in., which is 0.53% of the cross-sectional area of the catheter inner diameter of 0.00608 sq. in.

For metallic reinforcement layers containing only 1-4 wires, wire width and thickness are not restricted, however, in the distal region 102, the wire spacing Sₖ must be between 3-5 times the wire width wₖ and the wire angle θₖ must be less than 8 degrees.

While maintaining the requirements above, the Wire Coverage Ratio for the present invention should be 50-60% in at least the distal region 102. In one example, a catheter body 100 with an inner diameter of 0.088″ has three (3) Parallel Groups of metallic reinforcement. The first Parallel Group can be the layer 150 and comprised of a single 0.002″ round wire with a spacing sₖ of 0.0065″ and a wire angle θₖ of 1.3 degrees in the distal region 102. The second Parallel Group can be comprised of eight wires of 0.001″×0.002″ flat wire (wire width is 0.002″) with a spacing Sₖ of 0.010″ from one wire to the next and a wire angle θₖ of 15 degrees in the distal region 102, wound clockwise. The third Parallel Group can also be comprised of eight wires of 0.001″×0.002″ flat wire (wire width is 0.002″) with a spacing sₖ of 0.010″ from one wire to the next and a wire angle θₖ of 15 degrees in the distal region 102, but wound counterclockwise and inter-woven with the second Parallel Group to create a single layer 160. Therefore, the Wire Coverage Ratio (WCR) by all three Parallel Groups (two Layers with 17 total wires) is 56.5% in the distal region 102.

A first metallic reinforcement layer 150 which is comprised of a single parallel group and only 1 to 4 wires provides significant kink resistance and hoop strength for the catheter body 100, without adding significant stiffness. A second metallic reinforcement layer 160 comprised of two parallel groups which are inter-woven as described above provides torque resistance, tensile strength, and supports additional kink resistance and hoop strength for the catheter body 100. Utilizing a small Wire Size Ratio and large Wire Coverage Ratio in the distal region 102, as described above, allows the second metallic reinforcement layer 160 to provide these aforementioned benefits without significantly increasing the bending stiffness of the catheter body 100 in the distal region 102, resulting in a highly navigable but durable neurovascular catheter.

The WCR, WSR, wire angle θₖ, spacing sₖ, number of wires, wire cross-sectional area (including with width wₖ and wire thickness), number of reinforcement layers, or parallel groups n may be modified beyond the distal region 102 (i.e., more proximally on the catheter body 100 than the distal region 102 which comprises the distal 3 cm-30 cm of the catheter body 100) without impacting the performance in the distal region 102, although the same parameters can also be used for the length of the catheter body 100.

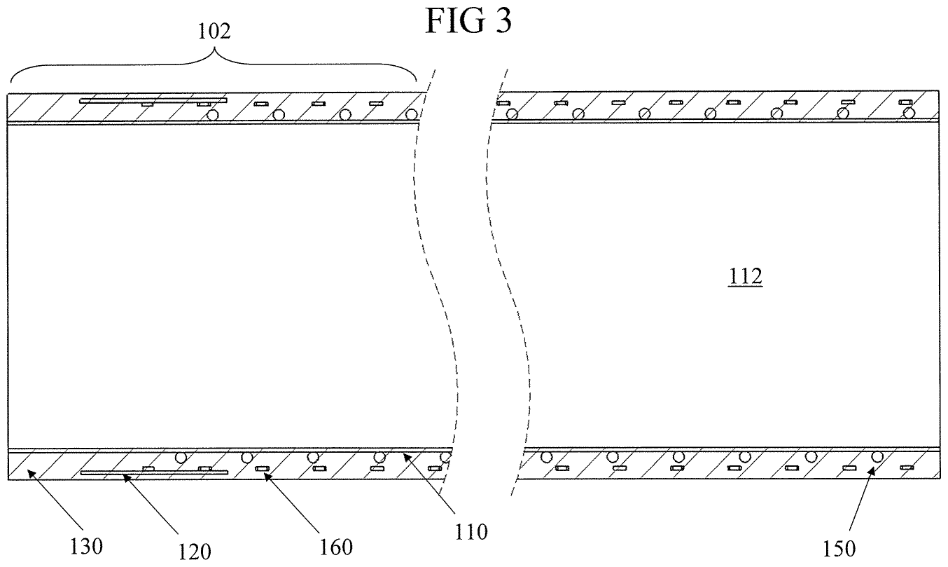

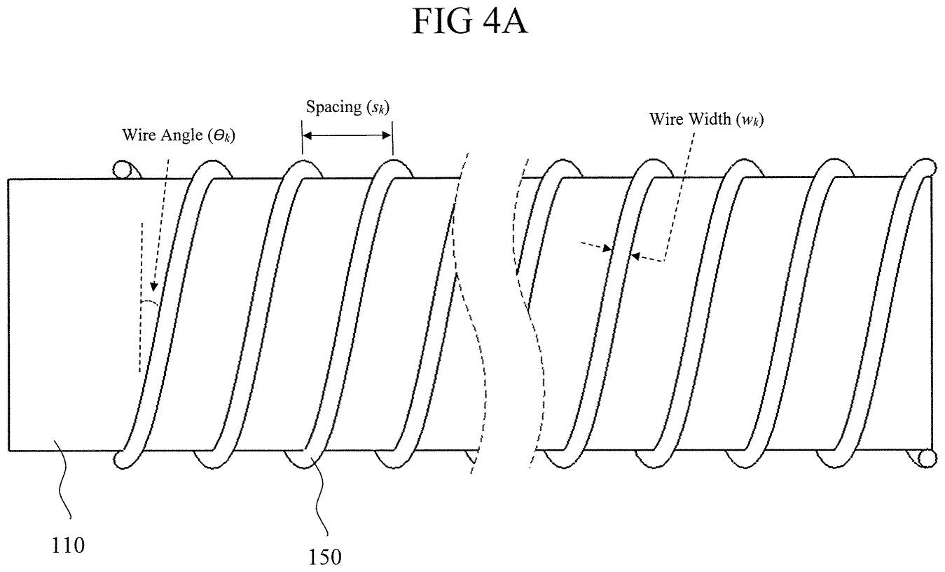

The marker band 120 is a tubular radiopaque marker which has a distal end, a proximal end, and an inner opening, and is a metal or alloy which provides visibility under fluoroscopy such as gold, platinum, or a 90% Platinum/10% Iridium alloy. The inner opening of the marker band 120 is positioned around the layers of the reinforcement structure 140, with the distal end of the marker band positioned between 0.5 mm-1.0 mm from the distal end of the catheter. The distal ends of the layers for the reinforcement structure 140 fall between the proximal end of the marker band 120 and the distal end of the catheter, but preferably between the proximal end of the marker band 120 and the distal end of the marker band 120. See FIG. 3. The marker band 120 is fused to the layers of the reinforcement structure 140 by the addition of heat or energy to weld the components together, but never by the addition of material such as adhesive or solder, which may increase the stiffness and affect the bending profile of the assembly.

The outer jacket 130 is comprised of varying durometers of thermoplastics which are melted under compression onto the underlying reinforcement structure 140 and the inner liner 110 to create the composite wall by encapsulating the reinforcement structure 140 and the marker band 120, while also bonding to the outer surface of the inner liner 110 to create the catheter assembly. Softer and more flexible polymers comprise the distal end of the outer jacket 130 while harder and more rigid polymers comprise the proximal end of the outer jacket 130, thereby creating a catheter shaft with a varying stiffness profile which typically is softest at the distal end and gradually becomes more stiff moving proximally along the shaft. This creates a stiff and supportive composite on the proximal end which allows the user to easily control the device, while simultaneously creating a flexible distal end that is capable of navigating the tortuous vasculature of the cerebral circulation.

In this regard, the present invention is also directed to catheters utilizing relatively soft plastic materials, preferably relatively soft thermoplastic elastomers, at the distal tip to achieve improved catheter performance. Such plastics and elastomers preferably have hardness durometers less than 80 Shore A (hereinafter “soft elastomer”), and can include Polyether block amides, polyurethanes, and silicones, and preferably have a distal tip wall thickness of 0.003″-0.012″, and a length of 10 mm or more, as measured along the longitudinal axis of the catheter. The plastic materials for other sections of the catheter can be known plastics, such as polyurethane, Polyether block amide, nylon, polycarbonate, or polyamide.

In one embodiment, the distal-most (first) portion of the outer jacket 130 is a soft elastomer having hardness 40-55 Shore A and can be 2-8 cm but preferably 3 cm. A second portion from the distal end is a soft elastomer having hardness 50-65 Shore A and can be 1-3 cm but preferably 2 cm. A third portion from the distal end is a soft elastomer having hardness 60-80 Shore A and can be 2-5 cm but preferably 3 cm. A fourth portion is a thermoplastic having hardness 20-40 Shore D and can be 4-14 cm but preferably 14 cm. A fifth portion is a thermoplastic having hardness 25-45 Shore D and can be 3-8 cm but preferably 6 cm. Sixth through tenth portions are various thermoplastics having hardness 35-75 Shore D and can be 2-4 cm each but preferably 2 cm each. An eleventh portion is a Nylon 12 that accounts for the remaining length of the catheter.

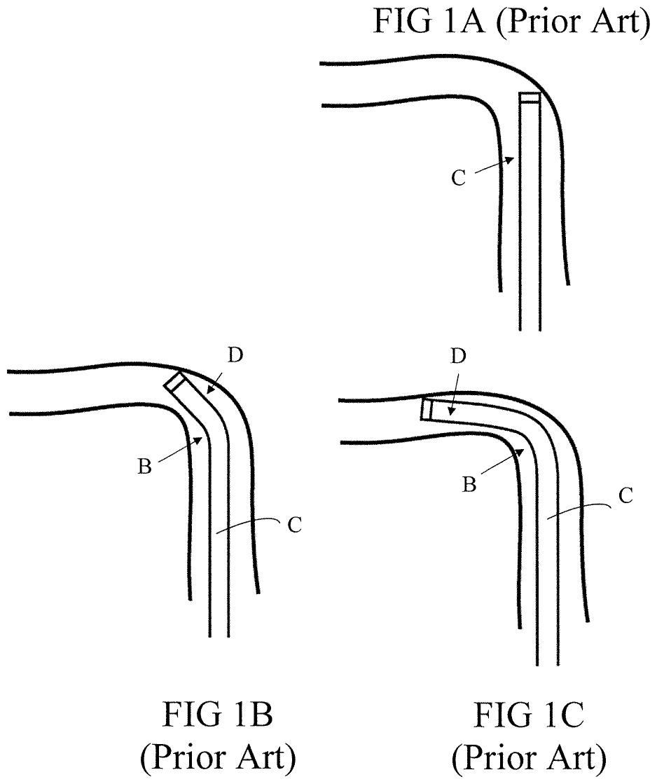

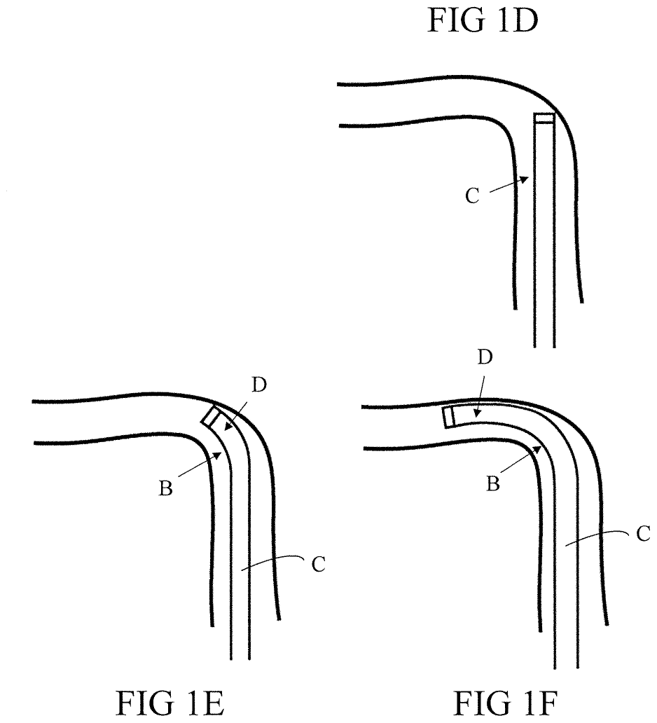

Utilizing a soft elastomer as the jacket material at the distal end of a catheter results in improved tip bending behavior, which is advantageous for navigation through curves in neurovascular arteries. The properties of the catheter are further improved if the jacket material is used in combination with structural and reinforcement materials to enable sufficient hoop strength, kink resistance, and collapse-resistance under vacuum. Referring to FIGS. 1D, 1E and 1F, the catheter is able to start bending almost immediately at the distal tip region when it reaches a bend in a vessel, which results in a smaller catheter bend radius and improved ability to navigate the catheter through tight bends and through small vessels, and to guide the catheter over a microcatheter or guidewire in a vessel, enabling the catheter to go farther into the brain, if used in a vessel in the brain.

In one embodiment, the catheter according to the present invention has the following dimensions at the distal end: 0.088″ inner diameter, 0.104″ outer diameter, and 0.008″ wall thickness.

The catheters of this disclosure would be used by physicians (such as interventional radiologists and neuro-interventionalists) during standard minimally invasive catheterization procedures.

Utilizing a soft elastomer as the jacket material at the distal end of a catheter in combination with the metallic reinforcement structure of the present invention results in a catheter tip that is also sufficiently atraumatic to permit intermediate and large-bore catheters (e.g., catheters with inner diameters between 0.040″-0.110″ and outer diameters between 0.045″-0.140″) to navigate into the distal neurovasculature, such as the middle cerebral artery in the anterior circulation of the brain and the vertebral, basilar, and posterior cerebral arteries in the posterior circulation of the brain. These sufficiently atraumatic tips are soft and flexible enough to conform to the vessel shape and not exert excessive force against the inner wall of the vessel thereby alleviating dissections and perforations.

Utilizing a soft elastomer as the jacket material at the distal end of a catheter in combination with the metallic reinforcement structure of the present invention results in improved kink resistance, elastic behavior, and flexibility.

The catheter of the present invention enables users to navigate more quickly and successfully around bends in vessels to the target site. In certain situations, the catheter can make its way through vessel bends that current catheters cannot, or have difficulty to, bend through. In other situations, the catheter of the present invention will reduce procedure time by moving its way through vessel bends more quickly than current devices.

Increased rates of successfully navigating to the target site result in more procedures being performed optimally. This decreases procedure times by more rapidly navigating the bends of the neurovasculature and results in less risk to patients and faster therapies. Sufficiently atraumatic tips will reduce adverse events, injuries, and complications related to the procedure.

This material and construction of the present invention enables a force reduction of 30-50% or greater during tip deflection when compared to existing catheters.

While the description above refers to particular embodiments of the present invention, it will be understood that many modifications may be made without departing from the spirit thereof. The accompanying claims are intended to cover such modifications as would fall within the true scope and spirit of the present invention.

1-14. (canceled)

15. A catheter body having a distal end and comprising:
a lubricious polymeric inner liner;
a composite wall comprising:
a reinforcement structure having a first layer of one or more metallic wires and a second layer of one or more metallic wires, with each layer of metallic wires wound in helical patterns, with the first layer of one or more metallic wires wound around the inner liner and the second layer of one or more metallic wires wound around the first layer of one or more metallic wires, wherein the first layer of one or more metallic wires and the second layer of one or more metallic wires are together comprised of at least three Parallel Groups of metallic wires; and
an outer jacket that is comprised of a distal jacket material that is a soft elastomer of hardness 60 Shore A or lower;
wherein all the layers of one or more metallic wires of the reinforcement structure together results in a Wire Coverage Ratio (WCR) which is the summation of the coverage percentage by each Parallel Group of wires, wherein “Parallel Groups” means all metallic wires that are wound in the same direction and at the same angle such that they never intersect, regardless of which layer they are in; and
wherein the catheter body has an inner diameter of 0.088″ with three Parallel Groups, where a first Parallel Group is the first layer and comprises a single wire with a spacing sₖ that is between 3-5 times the wire width wₖ and a wire angle θₖ that is less than 8 degrees, a second Parallel Group is comprised of eight wires of 0.001″×0.002″ flat wire with a spacing sₖ of 0.010″ from one wire to the next and a wire angle θₖ of 15 degrees, wound clockwise, and where a third Parallel Group is comprised of eight wires of 0.001″×0.002″ flat wire with a spacing sₖ of 0.010″ from one wire to the next and a wire angle θₖ of 15 degrees, but wound counterclockwise and inter-woven with the second Parallel Group to create a single layer; and wherein the WCR of the combined first Parallel Group, the second Parallel Group and the third Parallel Group is 50-60%.

16. The catheter body of claim 15, wherein WCR is defined by the equation:
$$W C {R}_{n} = {\sum }_{k = 1}^{n} ( \frac{{w}_{k}}{( cos {θ}_{k} ) ( {s}_{k} )} ) ( 100 \% - W C {R}_{( k - 1 )} )$$
Where:
n=number of distinct Parallel Groups of wires in the reinforcement structure
WCRₙ=The combined Wire Coverage Ratio of all n Parallel Groups of wire(s) of the reinforcement structure
wₖ=the wire width of wire(s) in the k^{th} Parallel Group
θₖ=the angle of wire(s) in the k^{th} Parallel Group, as measured relative to a cross-section of the catheter body that is parallel to an end of the catheter body
sₖ=the spacing between consecutive wires in the k^{th} Parallel Group.

17. The catheter body of claim 15, wherein the soft elastomer has a hardness 42 or 52 Shore A.

18. The catheter body of claim 15, wherein the catheter body has a distal region which extends for a length of 3 cm to 30 cm from the distal end of the catheter body, and wherein the WCR of 50-60% for all the layers of one or more metallic wires of the reinforcement structure is only at the distal region.

19. The catheter body of claim 15, further including a tubular marker band that has a distal end, a proximal end, and an inner opening, with the inner opening of the marker band positioned around the first layer of one or more metallic wires and the second layer of one or more metallic wires, and wherein the first layer of one or more metallic wires and the second layer of one or more metallic wires each has a distal end that is positioned between the proximal end of the marker band and the distal end of the catheter.

20. The catheter body of claim 15, further including a tubular marker band that has a distal end, a proximal end, and an inner opening, with the inner opening of the marker band positioned around the first layer of one or more metallic wires and the second layer of one or more metallic wires, and wherein the first layer of one or more metallic wires and the second layer of one or more metallic wires each has a distal end that is positioned between the proximal end of the marker band and the distal end of the marker band.

21. The catheter body of claim 15, wherein the outer jacket is provided with softer polymers closer to the distal end of the catheter body and with harder and more rigid polymers proximal from the distal end of the catheter body.

22. The catheter body of claim 15, wherein the spacing sₖ of the first Parallel Group is 0.0065″ and the wire angle θₖ for the first Parallel Group is 1.3 degrees.

23. The catheter body of claim 15, wherein WCR is 56.5%.
